(19) United States
(12) Patent Application Publication
(10) Pub. No.: US 2011/0195974 A1
(43) Pub. Date: Aug. 11, 2011

(54) METHODS OF TREATING INFLAMMATORY CONDITIONS WITH ADRENERGIC ANTAGONISTS

(76) Inventor: Rekha Bansal, Twinsburg, OH (US)

(21) Appl. No.: 12/674,818

(22) PCT Filed: Aug. 25, 2008
(86) PCT No.: PCT/US08/74212

§ 371 Date: Apr. 29, 2011

Related U.S. Application Data
(60) Provisional application No. 60/957,963, filed on Aug. 24, 2007.

(51) Int. Cl. A61K 31/517; A61K 31/4375; A61K 31/4164; A61P 29/00; A61P 19/00; A61P 11/00; A61P 3/00; A61P 17/00; A61P 25/28; A61P 9/00; A61P 31/18

(52) U.S. Cl. 514/252.17; 514/280; 514/401

(57) ABSTRACT

A method of treating inflammation and joint deterioration in mammals includes administering a therapeutically effective dose of alpha 1A receptor antagonists alone or in combination with a beta 2 adrenergic antagonists or beta 2 adrenergic antagonists and beta 2 adrenergic agonists.

### RELATED APPLICATION

This application claims priority from U.S. Provisional Application No. 60/957,693, filed Aug. 23, 2007, the subject matter, which is incorporated herein by reference.

### FIELD OF THE INVENTION

The present invention provides a method for inhibiting tumor necrosis factor-alpha (TNF α) release as a way of treating autoimmune inflammatory diseases. The present invention also relates to a method of treating cardiac related disorders by inhibiting TNF.

### BACKGROUND OF THE INVENTION

TNF α is a potent immuno-mediator and pro-inflammatory cytokine that has been implicated in the pathogenesis of a number of human diseases. In clinical situations, an immediate local release of TNF α from cells at or adjacent to the injury site by chemotactic cytokines is common. Activated macrophages are a major cellular source for TNF α, although other cell types such as T-cells, mast cells, neutrophils, endothelial cells, microglia, and astrocytes can be stimulated to secrete TNF α.

TNF α also plays a role in the initiation of inflammation and the resulting pathophysiologic consequences of inflammation. TNF α causes an elevation of IL-1, stimulation of neutrophil degranulation, and enhancement of phagocytosis.

Over production of TNF α is closely linked to the development of many autoimmune and acute clinical conditions including: rheumatoid arthritis, rheumatoid spondylitis, osteoarthritis, other arthritic conditions, sepsis (gram-negative sepsis), septic-shock endotoxic shock (toxic shock syndrome), adult respiratory distress syndrome, chronic pulmonary inflammatory disease, silicosis, asbestosis, pulmonary sarcoidosis, bone resorption diseases, graft vs. host reactions, allograft rejections, immune deficiency syndrome (AIDS), keloid formation, scar tissue formation, Crohn's disease, fibromyalgia, ulcerative colitis, or pyresis, Multiple Sclerosis, autoimmune diabetes, systemic lupus erythematosus, asthma, xeno transplantation, chronic bronchitis, atopic dermatitis, urticaria, allergic rhinitis, allergic conjunctivitis, eosinophilic granuloma, reperfusion injury of the myocardium and brain, chronic glomerulonephritis, alzheimer's disease, pulmonary fibrosis, lung sarcoidosis, hepatic apoptosis, obesity, pre-eclampsia, dermal burns, cardiac arrest, congestive heart failure, myocardial infarction, acute allogenic bone transplants, and HIV viral infections.

TNF α and IL1 beta remain important in pathophysiology of acute and chronically harmful diseases. TNF-α is known to stimulate release of: interleukin-1, interleukin-6, interleukin-8, platelet-activating factor, leukotrienes, thromboxane A2, and prostanglandins. It may stimulate macrophages directly to promote its own release. TNF promotes adhesion of endothelial cells, polymorphonuclear cells, eosinophils, basophils, monocytes, and occasionally, lymphocytes by inducing increased expression of adhesion molecules. By an indirect mechanism, TNF has the potential of activating a common pathway of coagulation and complement system.

Since TNF α is an important mediator of many reactive pathophysiologic states or diseases, inhibitors of TNF α production (synthesis and release) can have utility in any patho-physiologic state or disease with abnormally high levels of TNF α. TNF α antagonists have been shown effective in treating disorders that follow cellular tissue or organ injury. The discovery of compounds that inhibit TNF α overproduction provide a therapeutic approach for those diseases in which excessive or unregulated TNF α tissue levels is implicated.

Endotoxin from gram negative bacteria has been used to induce TNF α both in vivo and in vitro. In addition to endotoxin, certain micro-organisms as well as other substances, such as IL-1, are capable of inducing secretion of TNF α from stimulated peritoneal macrophages. In pathological conditions, unregulated TNF α production by monocytes, macrophages or related cells are implicated in exacerbating and/or causing the disease. TNF α, a pleiotropic cytokine, is produced, stored or released by macrophages and other cells as an initial response when exposed to a variety of infections or non-infectious disorders. Excessively high blood or tissue levels of TNF α are often lethal to cells, tissues, organs, and the host. TNF α is secreted in response to stimulation by lipopolysaccharides (LPS), viruses, bacteria, parasites, antigens, and allograft rejection among other injuries.

### SUMMARY OF THE INVENTION

The present invention discloses a method for the treatment of inflammatory conditions in a subject by administering alpha 1A adrenergic antagonist and its derivatives/analogs alone, or in combination with a beta 2 adrenergic antagonist or a beta 2 adrenergic antagonist and a beta 2 adrenergic agonist. The inflammation can be local or systemic.

In accordance with an aspect of the invention, the alpha 1A adrenergic receptor antagonist does not lower the blood pressure of the mammal The alpha 1A adrenergic receptor antagonist can prevent detectable TNF production and or release and can inhibit IL-1 production and/or release.

The present invention also provides a new use of the alpha 1A adrenergic receptor antagonist compounds for treating rheumatoid arthritis, rheumatoid spondylitis, osteoarthritis, other arthritic conditions, sepsis (gram-negative sepsis), septic-shock endotoxic shock (toxic shock syndrome), adult respiratory distress syndrome, chronic pulmonary inflammatory disease, silicosis, asbestosis, pulmonary sarcoidosis, bone resorption diseases, graft vs. host reactions, allograft rejections, immune deficiency syndrome (AIDS), keloid formation, scar tissue formation, Crohn's disease, fibromyalgia, ulcerative colitis, or pyresis, Multiple Sclerosis, autoimmune diabetes, systemic lupus erythematosus, asthma, xeno transplantation, chronic bronchitis, atopic dermatitis, urticaria, allergic rhinitis, allergic conjunctivitis, eosiniophilic granuloma, reperfusion injury of the myocardium and brain, chronic glomerulonephritis, alzheimer's disease, pulmonary fibrosis, lung sarcoidosis, hepatic apoptosis, obesity, pre-eclampsia, dermal burns, cardiac arrest, congestive heart failure, myocardial infarction, acute allogenic bone transplants, and HIV viral infections. In one example, the alpha 1A adrenergic antagonist can prevent joint inflammation and damage.

Therapeutic benefit can be achieved by treating patients with a therapeutically effective amount of an alpha 1A-adrenergic antagonist alone (e.g., prazosin and/or prazosin derivatives) or in combination with a beta 2 adrenergic antagonist (e.g., butoxamine) or a beta 2 adrenergic antagonist and a beta 2-adrenergic agonist (e.g., salbutamol). Therapeutically effective ratios of each drug in combination therapy and frequency of treatment with the compound can be variable and can be determined by those skilled in the art.

Alpha A1 adrenergic antagonists of the invention along with beta 2 adrenergic antagonists and beta 2 adrenergic agonists may be administered to a patient in therapeutic or sub-therapeutic dosages by themselves (e.g., neat). Alpha Al adrenergic antagonists, beta 2 adrenergic antagonists, and beta 2 adrenergic agonists can also be administered in the form of pills, tablets, capsules, caplets, solutions, suspensions, syrups, suppositories, and aerosols. The route of administration of the compound may be include sublingual, oral, intravenous, intramuscularly, rectal, parenteral, or subcutaneous administration. The compounds of the invention may also be in salt form.

In one embodiment, therapeutically effective doses for alpha 1A adrenergic antagonist range, respectively, from about 0.001 to 100.0 mg/kg. Depending on disease severity and patient responses to the drugs, the compound may be administered three times daily in therapeutically effective dosages. Therapeutically effective dosages of alpha 1A adrenergic and beta 2 adrenergic antagonists in combination (e.g., prazosin and butoxamine) or alpha 1A adrenergic, beta 2 adrenergic antagonists, and beta 2 adrenergic agonists (e.g., prazosin, butoxamine, and salbutamol) can range from about 0.001 to about 100.0 mg and be administered two or more times per kg/day.

### BRIEF DESCRIPTION OF THE DRAWINGS

FIG. 1 illustrates a graph showing the effect of LPS dose on TNF release in plasmalyte solvent. LPS directly affects TNF release, as is clearly shown in FIG. 1. Blood dilutions 50%, 40%, 20%, 10%, and 5% were treated with various doses of LPS and incubated at 37° C. for 2 h. The blood was spun down at 1300 g, and the plasma was separated for TNFα ELISA. This assay was conducted in duplicates to determine what concentration of LPS would generate a detectable TNF signal. Maximum TNF signal was obtained at LPS doses from 1 μg/ml to 60 μg/ml. The dose response of blood concentrations were linear in the range of blood dilutions tested. The bottom most line shows buffer alone. From top to bottom, concentrations were 50%, 40%, 20%, 10%, and 5%.

FIG. 2 illustrates the effect of various LPS dosages on blood dilution. LPS at 10 μg/ml causes a dose dependent production of TNF in whole blood assay. Blood at various dilutions (50%, 40%, 20%, 10%, and 5%) were treated with a constant dose of LPS. ELISA data shows that the TNF production was linearly proportional to blood concentration. A similar dose curve was obtained with 1-2 μg/ml of LPS.

FIG. 3 illustrates a graph showing the inhibition of LPS-induced TNFα release by alpha adrenergic antagonists, prazosin, yohimbine, and phentolamine. This assay evaluated the ability of α-adrenergic antagonists to inhibit the production or release of TNFα. These data are evaluated using an LPS concentration of 10 μg/ml in 25% blood diluted in plasmalyte. These data are a fitted representation of our dose curves for the above-mentioned drugs. The experiment was conducted at least 2 times in duplicates and the fitted representation above consists of the average of those experiments. The IC50 values were calculated using logistic analysis on Origin Software.

FIG. 4 illustrates a graph showing the inhibition of release of TNF α by terbutaline and salbutamol. This assay evaluated the inhibition of TNF production by salbutamol and terbutaline using the whole blood assay. This method utilized an LPS induction at 10 μg/ml in 25% whole blood diluted in plasmalyte. This data demonstrates that effectiveness of salbutamol and terbutaline is, at best, 50% at the upper range of the dose curve. Higher doses were not evaluated because the equivalent mg/kg doses would be too high, therefore, limiting their practical use under therapeutic conditions. IC50 values were not calculated. There is not a variation of doses to suggest that IC50 is available. The data and the fit were calculated using Origin Software.

FIG. 5 illustrates a graph showing the inhibition of TNF production by butoxamine, and induction of TNF release by atenolol and metoprolol. We evaluated the affects of three beta-adrenergic antagonists for their inhibition in the production of TNF in 25% whole blood diluted in plasmalyte. We found very little inhibition if at all with butoxamine, and significant elevation in the TNF response with atenolol and metoprolol. Lack of significant TNF release by butoxamine in this assay and downregulation of inflammation arthritis suggests a mechanism other than TNF for the treatment of arthritis. Regardless of what the mechanism is, the affect of butoxamine and prazosin together in combination therapy was better than prazosin alone.

FIG. 6 illustrates a graph showing a combination of prazosin, prazosin+salbutamol, prazosin+salbutamol+butoxamine, prazosin+butoxamine, and prazosin+butoxamine+salbutamol. This assay evaluated the TNF inhibitory activity of prazosin in combination with other compounds at equal μg/ml dosing. This assay measured prazosin alone, and the combination of: prasozin/butoxamine, prasozin/butoxamine/terbutaline, prasozin/butoxamine/salbutamol, and prazosin/salbutamol. The data show a decrease in IC50 when prazosin is combined with one other drug, than compared to prazosin alone. When prazosin is in combination with 2 other drugs, the IC50 shifts further left.

FIGS. 7A and 7B illustrate the effect of an alpha 1A receptor antagonist Prazosin on Adjuvant Induced Arthritis in Lewis Rats. At day 16, the animals were started at a dose of Prazosin using intravenous and oral dosing. In this experiment, Lewis rats were purchased from Harlan Sprague Dawley. Each rat was weighed and kept in quarantine for 72 hours. Baseline Paw volumes were recorded using water displacement method for each rat. At day 16, all animals were weighed, anesthetized and right and left paw volumes were measured. Daily dosing started at Day 16; animals were grouped into non-diseased, diseased and treated. Indomethacin was used as a positive control. Each group consisted of five animals each. Drug treatments were given i.p or oral. The drug was used at 7.5 mg per kg (1.5 mg/200 g. per rat). As noted in the figure, the drug prevents inflammation in arthritis y nearly 50% level. The amount of inhibition seen with Prazosin alone is in conformity with the FDA approved drugs. As shown in FIGS. 7A and 7B, Prazosin effect is visible for both paws.

FIG. 8 illustrates plots comparing the mean clinical arthritis score per day for collagen induced arthritis (CIA) mice administered Prazosin, Enbrel, and Control. The dark black square is disease animals. The purple squares are Enbrel, Cyan squares are Prazosin and open squares are non-disease (n=6). Higher “n” should give smaller error bars.

FIG. 9 illustrates the body change of CIA mice administered Prazosin, Enbrel, and Control. Compared to non disease the animals did not lose weight. The weight change with respect to day 0 is essentially unchanged for disease, Enbrel, and NM2014 group.

FIG. 10 illustrates a graph showing that Prazosin inhibits TNF release in a human whole blood assay.

FIG. 11 illustrates photographs comparing knee joint damage of CIA mice administered Prazosin and Enbrel.

FIG. 12 illustrates photographs comparing ankle joint integrity in CIA mice administered Prazosin and Enbrel.

### DETAILED DESCRIPTION OF THE INVENTION

For convenience, certain terms employed in the specification, examples, and appended claims are collected here. Unless defined otherwise, all technical and scientific terms used herein have the same meaning as commonly understood by one of ordinary skill in the art to which this invention belongs.

As used herein, the term “subject” refers to any warm-blooded organism including, but not limited to, human beings, pigs, rats, mice, dogs, goats, sheep, horses, monkeys, apes, rabbits, cattle, etc.

As used herein, the terms “treatment,” “treating,” or “treat” refers to any specific method or procedure used for the cure of, inhibition of, prophylaxis of, reduction of, elimination of, or the amelioration of a disease or pathological condition (e.g. arthritis) including, for example, preventing inflammation associated with arthritis from developing, inhibiting inflammation development, arresting development of clinical symptoms associated with inflammation, and/or relieving the symptoms associated with inflammation.

As used herein, the term “effective amount” refers to a dosage of an alpha 1A adrenergic receptor antagonist administered alone or in combination with with a beta 2 adrenergic antagonist (e.g., butoxamine) or beta 2 adrenergic antagonist and beta 2-adrenergic agonist (e.g., salbutamol) and any additional therapeutic agents that are effective and/or sufficient to provide treatment of inflammation and/or a disease or disorder associated with inflammation and/or TNF production or release. The effective amount can vary depending on the subject, the disease being treated, and the treatment being effected.

As used herein, the term “therapeutically effective amount” refers to that amount of a an alpha 1A adrenergic receptor antagonist administered alone or in combination with a beta 2 adrenergic antagonist (e.g., butoxamine) or beta 2 adrenergic antagonist and beta 2-adrenergic agonist (e.g., salbutamol) that results in amelioration of symptoms associated with inflammation and/or a disease or disorder associated with inflammation or TNF production and/or results in therapeutically relevant effect. By way of example, a “therapeutically effective amount” may be understood as an amount of alpha 1a adrenergic receptor antagonist required to reduce inflammation and/or TNF production in a subject.

As used herein, the terms “parenteral administration” and “administered parenterally” refers to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the terms “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

As used herein, “Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

The present invention relates to the use of small molecule adrenergic compounds that prevent TNF α production in post LPS stimulated monocytes. Monocytes/macrophages play an important role in an inflammatory cascade. In response to LPS challenge, monocytes/macrophages release pro-inflammatory cytokines. Similar to LPS there are several stimulants of TNF released from monocytes. Elevated circulating TNF α levels are associated with several acute and chronic inflammatory conditions such as: rheumatoid arthritis, rheumatoid spondylitis, osteoarthritis, other arthritic conditions, sepsis (gram-negative sepsis), septic-shock endotoxic shock (toxic shock syndrome), adult respiratory distress syndrome, chronic pulmonary inflammatory disease, silicosis, asbestosis, pulmonary sarcoidosis, bone resorption diseases, graft vs. host reactions, allograft rejections, immune deficiency syndrome (AIDS), keloid formation, scar tissue formation, Crohn's disease, fibromyalgia, ulcerative colitis, or pyresis, Multiple Sclerosis, autoimmune diabetes, systemic lupus erythematosus, asthma, xeno transplantation, chronic bronchitis, atopic dermatitis, urticaria, allergic rhinitis, allergic conjunctivitis, eosiniophilic granuloma, reperfusion injury of the myocardium and brain, chronic glomerulonephritis, alzheimer's disease, pulmonary fibrosis, lung sarcoidosis, hepatic apoptosis, obesity, pre-eclampsia, dermal burns, cardiac arrest, congestive heart failure, myocardial infarction, acute allogenic bone transplants, and HIV viral infections.

One aspect of the invention relates to a method of treating inflammation, an inflammatory condition, and/or disorders associated with TNF production in a subject by administering a therapeutically effective amount of an alpha 1A adrenergic receptor antagonist to the subject. The inflammation and/or inflammatory condition can be associated with TNF α production and the alpha 1A adrenergic receptor antagonist can be administered to subject in an amount effective to inhibit TNF α without lowering the blood pressure of the subject.

In one aspect of the invention, the method of treating inflammation does not include administering an alpha 2 adrenergic receptor antagonist and/or an alpha 1B adrenergic receptor antagonist to the subject. Alpha 1A adrenergic receptor antagonists that are specific to the alpha 1A adrenergic receptor and not to the alpha 1B and/or alpha 2 adrenergic receptor can be administered to subject to treat inflammation in accordance with the invention. Alpha 1B antagonists are involved in lowering the blood pressure and therefore it is desirable that an alpha 1B antagonist not be administered to the subject to treat inflammation.

Alpha 1A andrenergic receptor antagonists (i.e, alpha 1A antagonists) that are specific to the alpha 1A adrenergic receptor can include, for example, prazosin, doxazosin, tamsulosin, terazosin, phenoxybenzamine, 5-methyl urapidil, chloroethylclonidine, bunazosin, alfuzosin, RS17053, L-765,314, nicergoline, ABT-866, cyclazosin, A322312, A 119637, fiduxosin, JTH-601, WB4101, niguldipine, KMD3213, and UIC 14304. In a specific example of the present invention, the alpha 1A antagonist can comprise prazosin.

Other examples of alpha 1A antagonists are listed in U.S. Pat. No. 5,807,856, which is herein incorporated by reference in its entirety, and may be chosen from the list of compounds used for treating benign prostatic hyperplasia. The compounds of the present invention should be selective for alpha 1A and not alpha 1B. Still other example of alpha 1A antagonists that are selective or specific for alpha 1A and not alpha 1B are listed in U.S. Pat. Nos. 6,894,052, 6,890,921, 6,593,474, 6,399,614, 6,358,959, 6,124,319, 6,071,915, 5,661,163, 5,620,993, 5,403,847, 4,760,071, and 4,110,449, all of which are herein incorporated by reference in their entirety.

In another aspect of the invention, the method of treating inflammation, an inflammatory condition, or disorders associated TNF production can include administering a therapeutically effective amount of an alpha 1A antagonist in combination with a beta 2 adrendrenergic receptor antagonist. Beta 2 adrenergic antagonists (beta 2 adrenergic antagonist) can at least partially prevent TNF production. Beta 2 antagonists can include, for example, butoxamine (e.g., DL-erythro-α-(2,5-dimethoxyphenyl)-β-t-butyl aminopropanol hydrochloride, which is commercially available from GlaxoSmith Kline, N.C.). Other examples of beta 2 antagonists include ICI 118,551, H35/25, prenaterol, various 4- and 5-[2-hydroxy-3-(isopropylamino)propoxy]benzimidazoles, 1-(t-butyl-amino-3-ol-2-propyl)oximino-9 fluorene and various 2-(α-hydroxyarylmethyl)-3,3-dimethylaziridines. Methods of synthesis, beta 2 selectivity ratios and various biologic and pharmacologic properties of these compounds are known, and reported in for example, J. Pharm. Pharmacol., 1988, 32(9), 659-660; J. Med. Chem., 22(2), 210-214 (1979); J. Med. Chem., 21(1), 68-72 (1978); J. Med. Chem. 20(12), 1657-62 (1977); and Br. J. Pharmacol. 60(3), 357-362 (1977), all of which are herein incorporated by reference. Other examples of beta 2 antagonists are disclosed in U.S. Pat. No. 4,908,387, which is herein incorporated by reference in its entirety.

In still another aspect of the invention, the method of treating inflammation, an inflammatory condition, or disorders associated TNF production can include administering a therapeutically effective amount of an alpha 1A antagonist in combination with a beta 2 antagonist and a beta 2 andrenergic receptor agonist (i.e., a beta 2 agonist). Beta 2 agonists may treat or control acute or chronic bronchial asthma, exercise-induced bronchospasm, bronchitis, emphysema, bronchiectasis, other obstructive pulmonary diseases, and as a uterine relaxant in premature labor. One example of a beta 2 agonist that can be used in accordance with the present invention is salbutamol. Salbutamol reduces the severity of collagen-induced arthritis by down regulating TNF and upregulating TNF β and IL-10. Another example of beta 2 agonist is terbutaline. Terbutaline is also shown to reduce disease severity in rat arthritis model by 50%. Other examples of beta 2 agonists include metaproterenol, albuterol, isoetharine, pributerol, bitolterol, ritodrine, and salmeterol.

The alpha 1A antagonists alone or in combination with the beta 2 antagonists or beta 2 antagonists and beta 2 agonists in accordance with the present invention can be used to treat inflammatory conditions and diseases including but not limited to rheumatoid arthritis, rheumatoid spondylitis, osteoarthritis, other arthritic conditions, sepsis (gram-negative sepsis), septic-shock endotoxic shock (toxic shock syndrome), adult respiratory distress syndrome, chronic pulmonary inflammatory disease, silicosis, asbestosis, pulmonary sarcoidosis, bone resorption diseases, graft vs. host reactions, allograft rejections, immune deficiency syndrome (AIDS), keloid formation, scar tissue formation, Crohn's disease, fibromyalgia, ulcerative colitis, or pyresis, Multiple Sclerosis, autoimmune diabetes, systemic lupus erythematosus, asthma, xeno transplantation, chronic bronchitis, atopic dermatitis, urticaria, allergic rhinitis, allergic conjunctivitis, eosiniophilic granuloma, reperfusion injury of the myocardium and brain, chronic glomerulonephritis, alzheimer's disease, pulmonary fibrosis, lung sarcoidosis, hepatic apoptosis, obesity, pre-eclampsia, dermal burns, cardiac arrest, congestive heart failure, myocardial infarction, acute allogenic bone transplants, and HIV viral infections.

The term “treat inflammation” is intended to include the administration of therapeutic agents of the present invention to a subject for purposes, which can include amelioration or prevention of an inflammatory condition or disease. Such treatment need not necessarily completely ameliorate the inflammatory condition or disease. Further, such treatment can be used in conjunction with other traditional treatments for reducing the inflammatory condition known to those of skill in the art.

The alpha 1A antagonists alone or in combination with the beta 2 antagonists or beta 2 antagonists and beta 2 agonists can be provided in the form of pharmaceutical compositions. The pharmaceutical compositions of the present invention can be administered to any animal that can experience the beneficial effects of the compounds of the invention. Foremost among such animals are humans, although the invention is not intended to be so limited.

The pharmaceutical compositions of the present invention can be administered by any means that achieve their intended purpose. For example, administration can be by parenteral, subcutaneous, intravenous, intraarticular, intrathecal, intramuscular, intraperitoneal, or intradermal injections, or by transdermal, buccal, oromucosal, ocular routes or via inhalation. Alternatively, or concurrently, administration can be by the oral route. Particularly preferred is oral administration. The dosage administered will be dependent upon the age, health, and weight of the recipient, kind of concurrent treatment, if any, frequency of treatment, and the nature of the effect desired.

In addition to the pharmacologically active compounds, the pharmaceutical preparations of the compounds can contain pharmaceutically acceptable carriers comprising excipients and auxiliaries that facilitate processing of the active compounds into preparations that can be used therapeutically. The pharmaceutical preparations of the present invention are manufactured in a manner that is, itself, known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Examples of excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders, such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as acetylcellulose phthalate or hydroxypropylmethylcellulose phthalate, are used. Slow-release and prolonged-release formulations may be used with particular excipients such as methacrylic acid—ethylacrylate copolymers, methacrylic acid—ethyl acrylate copolymers, methacrylic acid—methyl methacrylate copolymers and methacrylic acid—methyl methylacrylate copolymers. Dye stuffs or pigments can be added to the tablets or dragee coatings, for example, for identification or in order to characterize combinations of active compound doses.

Other pharmaceutical preparations that can be used orally include push-fit capsules made of gelatin, as well as soft, sealed capsules made of gelatin and a plasticizer such as glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds are preferably dissolved or suspended in suitable liquids such as fatty oils or liquid paraffin. In addition, stabilizers may be added.

Examples of formulations for parenteral administration include aqueous solutions of the active compounds in water-soluble form, for example water-soluble salts and alkaline solutions. Especially preferred salts are maleate, fumarate, succinate, S,S tartrate, or R,R tartrate. In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example sodium carboxymethyl cellulose, sorbitol, and/or dextran. Optionally, the suspension may also contain stabilizers.

The following examples provided are illustrative, but not limiting, of the method and compositions of the present invention. Other suitable modifications and adaptations of the variety of conditions and parameters normally encountered and obvious to those skilled in the art are within the spirit and scope of the invention.

### EXAMPLES

### Example 1

### Whole Blood Assay to Determine Quantitative Levels of TNF in Whole Blood.

We collected whole human blood in a polypropylene tube. Containing a final concentration of 10 units of heparin per ml of blood using gravity flow with 19-gauge needle. To prevent non-specific activation of cells, we used the blood within 30 minutes of draw. The blood was diluted with plasmalyte to 50%, 40%, 30%, 20%, and 10% concentrations. In a matrix based assay set up, each blood dilution was treated with various concentrations of LPS dosages ranging from 60 μg/ml, 10 μg/ml, 6 μg/ml, 1 μg/ml, 0.6 μg/ml, 0.1 μg/ml, and 0.06 μg/ml. The LPS treated blood was incubated at 37 degree in an incubator with continues rotation. Following the incubation, the blood was centrifuged and the plasma was evaluated for TNF quantification using the ELISA assay (BD Biosciences, Los Angeles, Calif.).

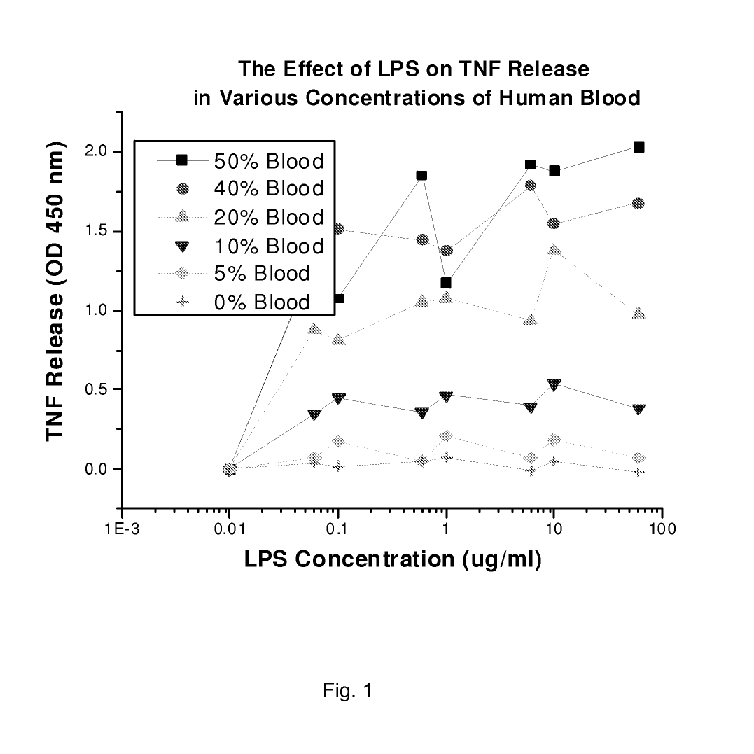

The results in FIG. 1, demonstrate a linear relationship between the concentrations of blood versus the release of measurable TNF. A readable OD was obtained at each of the blood dilutions treated with a constant dose of LPS at 10 μg/ml. This dose was found to give a maximum stimulation of TNF release from activated monocytes. The data suggests that a concentration of 25% blood concentration treated with 10 μg/ml of LPS displays an OD of 1.0 in the TNF assay. This is ideal to evaluate the effects of various drugs, alone or in combination.

### Example 2

### Optimization of TNF Whole Blood Assay

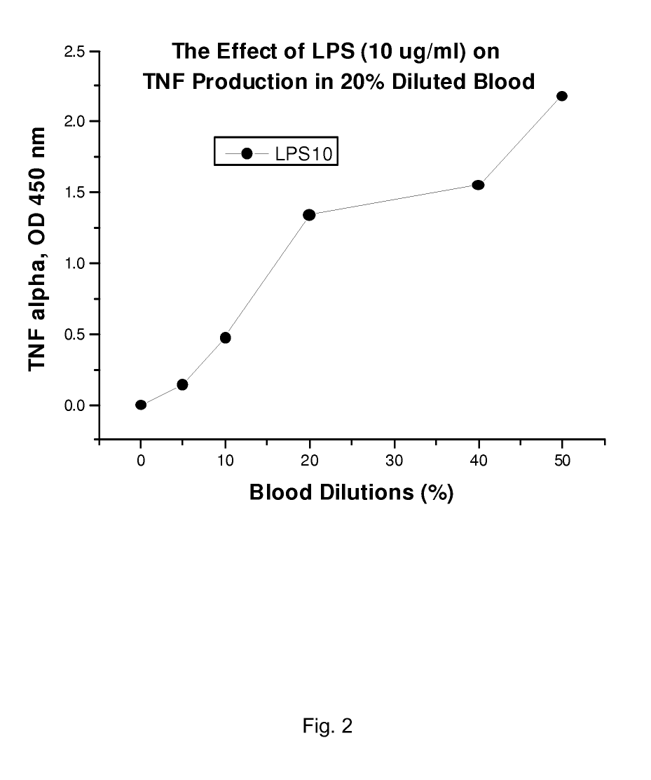

We initially tested the ability of LPS to produce TNF from monocytes, by adding various dilutions of LPS (60 μg, 10 μg, 6 μg, 1 μg, 0.6 μg, 0.1 μg, 0.06 μg) per ml of diluted blood (blood dilutions 50%, 40%, 20%, 10%, and 5%). Blood dilutions were made in the clinically relevant buffer “plasmalyte”. LPS treated blood was incubated at 37° C. for 2 hr. Following incubation, we separated the plasma samples by centrifugation. We evaluated the samples using TNF-ELISA. As shown in FIG. 1, the LPS dose at 10 μg/ml is ideal for production of TNF. Relying on FIG. 1, we conducted a blood dilution curve with 10 μg/ml LPS (FIG. 2). Based on interpretation of FIGS. 1 and 2, we concluded that blood dilution of 20-25% would be ideal at an LPS dose of 10 μg/ml. Based on the FIG. 2 data, we found LPS causes a near linear increase of TNF production in 20% blood. Based on these data, we decided to use 20-25% diluted blood and 10 μg/ml blood to saturate the activation of the blood to get a defined TNF signal in our ELISA assay. If there is too much blood, there will be heavy concentrations of TNF production, exceeding the detection levels of the assay. Using this optimization data, we created a fixed method to measure the inhibition of TNF production or release with various drugs and drug combinations.

### Example 3

### The Effect of α-Adrenergic Antagonists (Prazosin, Yohimbine, and Phentolamine) on TNF Inhibition

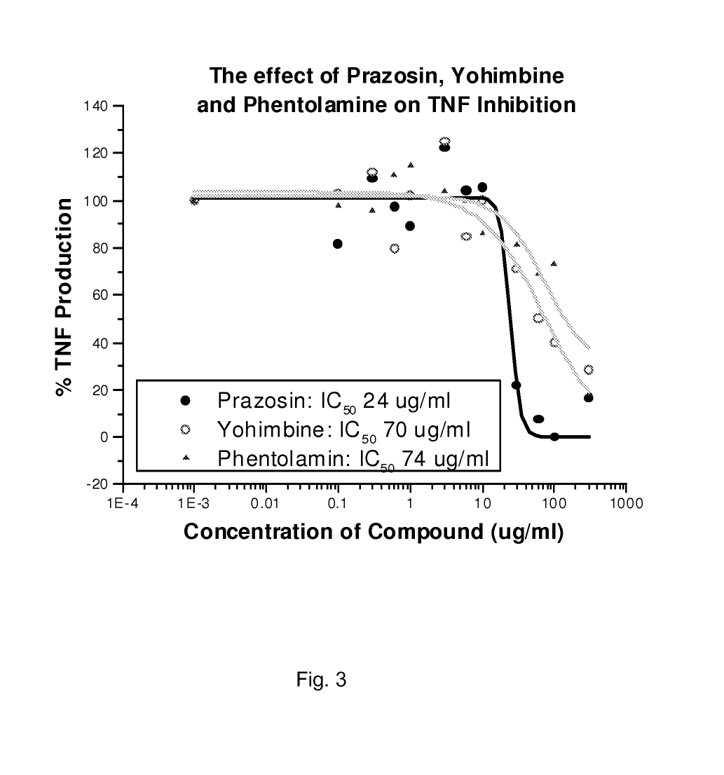

Using a whole blood assay, we tested the effects of the α-adrenergic antagonists (prazosin, yohimbine, and phentolamine) for the inhibition of TNF production in whole blood. Prazosin, an alpha 1A adrenergic antagonist, inhibited TNF at a low IC50 and demonstrated the lowest IC50 when compared with yohimbine (alpha 2A receptor antagonist) and phentolamine (alpha 1 and alpha 2 receptor antagonist). Prazosin inhibits TNF activity at 20 μg/ml drug concentration. Prazosin causes a definite inhibition of TNF α. Using prazosin treated samples, we observe nearly 100% inhibition at the upper range of the dose curve. These data suggest this compound can be used alone for the treatment of inflammation. It could also provide a possible treatment for rheumatoid arthritis. Yohimbine inhibited TNF with an IC50 in the 70-100μg/ml range (FIG. 3), with comparable potency to prazosin. Yohimbine (an alpha 2A antagonist) also demonstrates 80% inhibition of TNF production, or a release at the upper range of the dose curve. Despite its ability to inhibit TNF production, yohimbine has severe side effects that preclude its use as an approved treatment for RA. Phentolamine, a compound characterized as targeting both α-receptors, does not have the same level of inhibition as prazosin and yohimbine alone. Since phentolamine targets both receptors, it is possible that phentolamine would be a more potent compound. However, the data generated from this assay demonstrates otherwise. This leads us to believe that phentolamine inhibits TNF by binding to more than one receptor. However, from this data, we can demonstrate with confidence that targeted inhibition of the alpha 1A adrenergic receptor alone may be sufficient for the inhibition of TNF production, which is a significant target for the treatment of RA. Prazosin, an oral pill, is currently used for other clinical indications, and has an effect on hypertension. It is well known that arthritic patients are not hypotensive (CITE).

### Example 4

### The Effect of Beta-Adrenergic Agonists (Terbutaline and Salbutamol) on TNF Inhibition

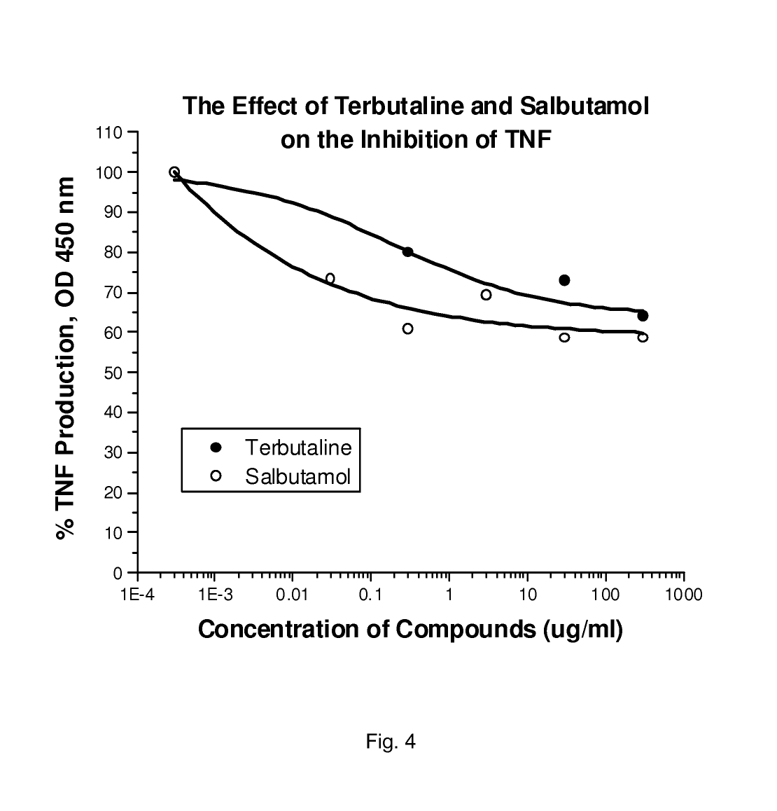

Both terbutaline and salbutamol inhibit inflammation by 50% in an arthritis model. We demonstrate that both drugs inhibit TNF production in our whole blood assay, as shown in FIG. 4. The dose of each drug causing maximum inhibition of TNF production is consistent with previous findings. Dose range studies showed terbutaline to have a minimal effect on the inhibition of TNF production. The degree of TNF inhibition corresponds to previously published data where terbutaline demonstrated a 50% benefit in mice with a collagen induced arthritis. Salbutamol demonstrates similar levels of inhibition of TNF at the upper range of the dose curve. This data corresponds well with previously reported data discussing the affect of salbutmol on TNF inhibition. The interesting point of note is the similarity in activities between salbutamol and terbutaline—being similar types of compound, salbutamol and terbutaline inhibit both TNF and inflammation in arthritis further supporting the idea that the inflammatory mechanism in arthritis is TNF based. The comparative analysis between the in vitro (whole blood TNF assay) and in vivo assays (arthritis animal studies) directly suggest the relevance of the whole blood assay with regard to inflammation. These data increase the confidence in the use of the whole blood assay as providing the direct relevance for developing anti-inflammatory compounds.

### Example 5

### The Effect of Aetnolol, Metoprolol, and Butoxamine on TNF Inhibition

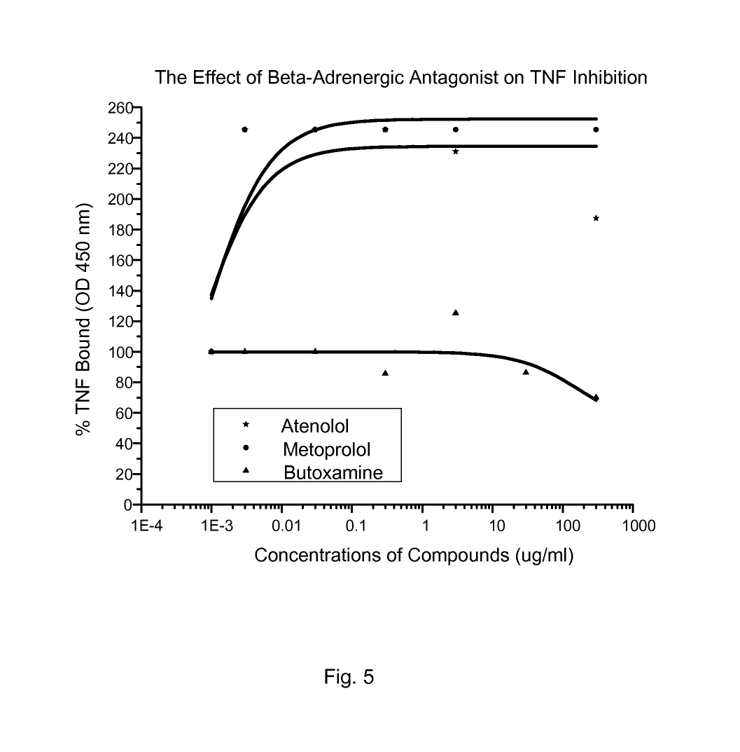

These drugs belong to the class of beta adrenergic receptors antagonists. However, the TNF assay differentiates these drugs into two categories: atenolol and metoprolol are activators of TNF release, butoxamine down regulates TNF production by 20%. Previous reports demonstrate that butoxamine is potent inhibitor of arthritis in animals. However, contrary to previous published reports, we found that butoxamine has a minimal effect in the inhibition on the production or release of TNF in blood. From our in vitro assay of TNF in whole blood, we found butoxamine to inhibit TNF at most approximately 20% at the upper range of the dose curve (FIG. 5). It is possible there may be complete inhibition at higher concentrations, but such doses could be toxic. The two other compounds, atenolol and metoprolol demonstrated a lack of TNF release inhibition, but actually increases exponentially TNF release into blood. These compounds showed an elevated TNF signal so high that the detection signal exceeded the detection sensitivity of our instruments. These compounds elevate the TNF response. Based on these studies, we can easily exclude atenolol and metoprolol for further evaluation, but butoxamine may have potential in the treatment of RA especially in combination with other drugs. While we are unclear regarding the actual mechanism of how butoxamine is inhibiting inflammation in animals, it is likely that the inhibition in animals may be via a different mechanism of IL-1 other than TNF.

### Example 6

### The Effect of Butoxamine and Salbutamol (Drug Combinations) on Prazosin Mediated TNF Inhibition

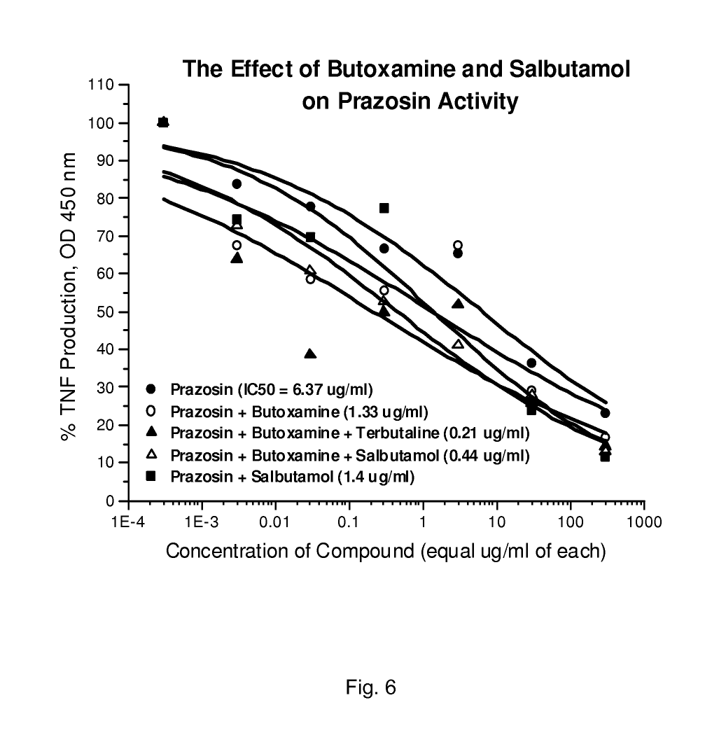

In these assays, we evaluated drug combinations at equivalent ug/ml. Prazosin was added in combination with other compounds in equal μg/ml concentrations (FIG. 6). When combined together with other drugs, the IC50 values shift by 5-10 fold. We see nearly complete inhibition at all doses.

### Example 7

### Effect of an Alpha 1A Receptor Antagonist Prazosin on Adjuvant Induced Arthritis in Lewis Rats

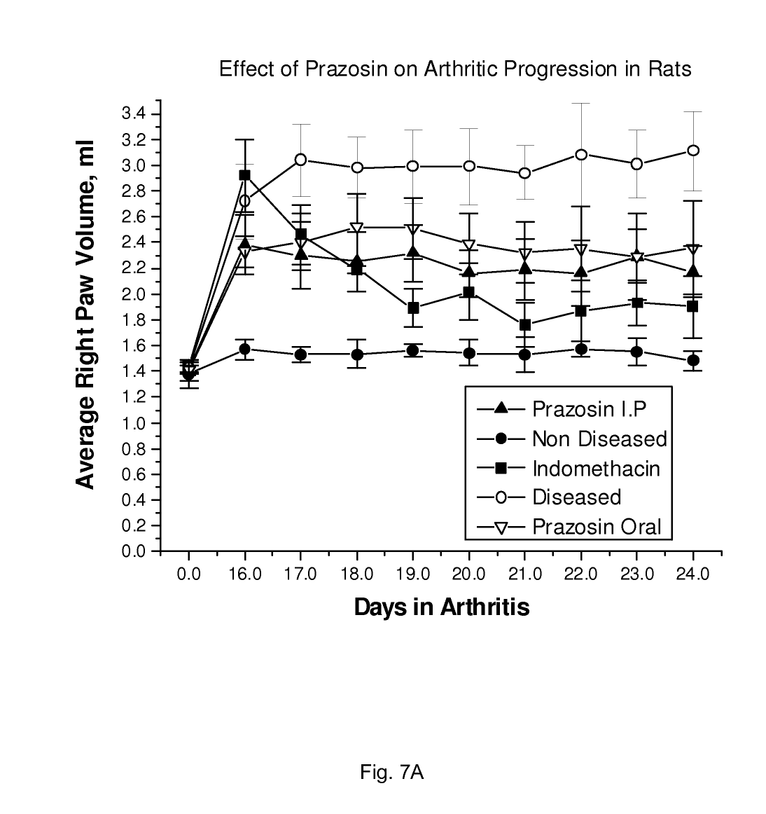

This study was designed to evaluate the effect of an alpha 1A adrenergic receptor antagonist “Prazosin” on Adjuvant Induced Arthritis is rats. Arthritis was induced with adjuvant. At day 16, the animals were started at a dose of Prazosin using intravenous and oral dosing. In this experiment, Lewis rats were purchased from Harlan Sprague Dawley. Each rat was weighed and kept in quarantine for 72 hours. Baseline Paw volumes were recorded using water displacement method for each rat. At day 16, all animals were weighed, anesthetized and right and left paw volumes were measured. Daily dosing started at Day 16; animals were grouped into non-diseased, diseased and treated. Indomethacin was used as a positive control. Each group consisted of five animals each. Drug treatments were given i.p. or oral. The drug was used at 7.5 mg per kg (1.5 mg/200 g. per rat). As noted in the figure, the drug prevents inflammation in arthritis at nearly 50% level. The amount of inhibition seen with Prazosin alone suggests that alpha 1A specific receptor antagonists can down regulate inflammation in AIA-rats. As shown in FIGS. 7A and 7B, Prazosin effect is visible for both paws.

### Example 8

### Prophylactic Mouse CIA Model of Arthritis

Male DBA/1J mice were given chicken type II collagen in Freund's complete adjuvant injections. Mice were randomized by body weight into treatment groups on study day 18. Treatment is initiated on study day 18 and continued once daily, every day (ip, qd). During this time clinical scores 0-5 were given to each of the four paws based on swelling. On days 21-35, the onset of arthritis occurred. Mice were weighed on days 18, 20, 22, 24, 26, 28, 30, 32, 34 and prior to tissue collection on day 35 (final day).

### Effect on Mouse Paw Inflammation Prazosin Reduces Paw Inflammation in Mouse Model of Collagen Induced Arthritis.

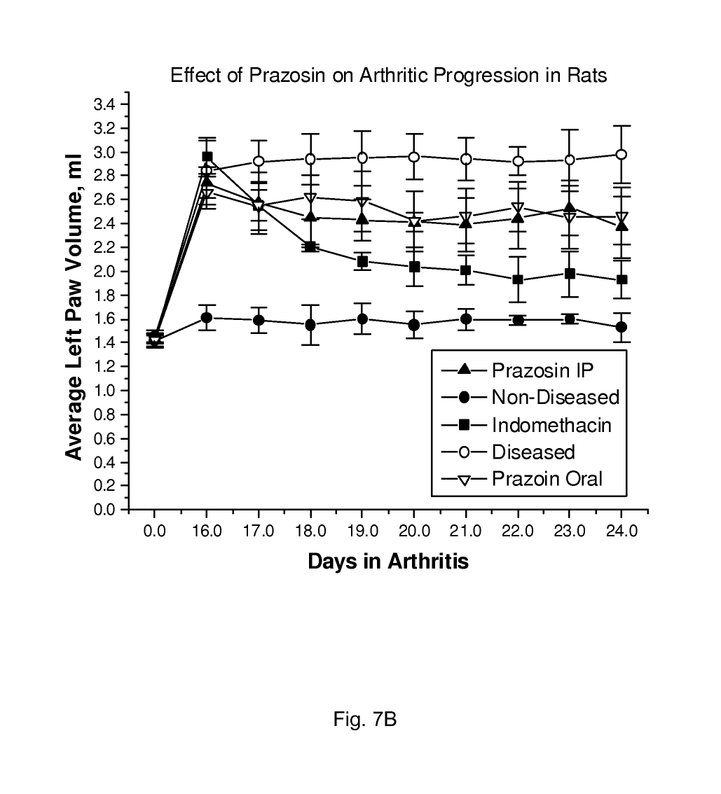

The treatment Prazosin was started at day 18. The drug was dosed daily at 1 mg/Kg/Daily. The data show that Prazosin reduces paw inflammation in mouse CIA. Compared to control, Prazosin significantly reduces swelling from day 25 on (p<0.001, Students t test) (FIG. 8). Enbrel is the current gold standard and is being used in arthritic patients. These experiments are very expensive, thus for our preliminary data we only evaluated 4 animals and have transposed this data over historical Enbrel data collected from this same animals species using the same experimental protocol. As can be seen, the effects of prazosin are clearly superior to the current gold standard for treating arthritis.

Effect on Prazosin on Body Weight: Weight is routinely assessed in the mouse model of CIA as a gross measure of drug toxicity.

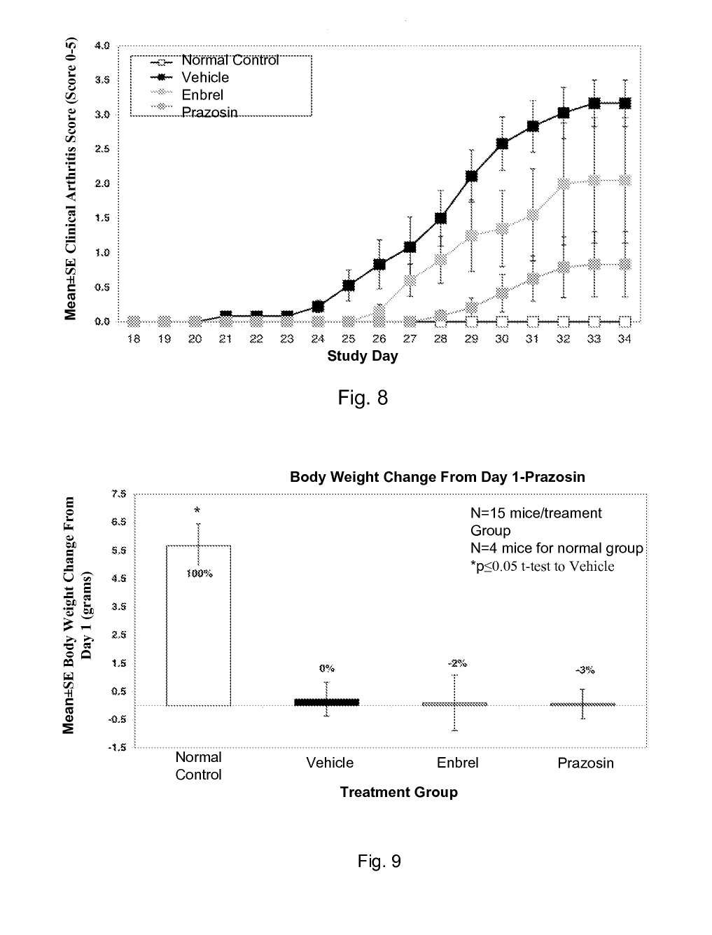

Untreated animals are expected to gain weight and collagen injected animals are expected to remain stable in weight (vehicle treated). Drug treated animals are expected to remain stable in weight if no overt toxicity of the drug treatment is present. Other manifestations of drug toxicity can always be present and are investigated at later stages of the preclinical drug development process, once efficacy in an animal model of the clinical disease is firmly established. In this set of experiments, weight remained constant (FIG. 9). The weight profile is similar to current gold standard of treatment and the vehicle treatment group. This is not surprising because prazosin is a commonly used FDA approved drug for indications other than arthritis and it is well known to not have any associated clinical toxicities at therapeutic doses in humans or mice.

### Example 9

### Prazosin Inhibits TNF Alpha Production in Thirteen Donors

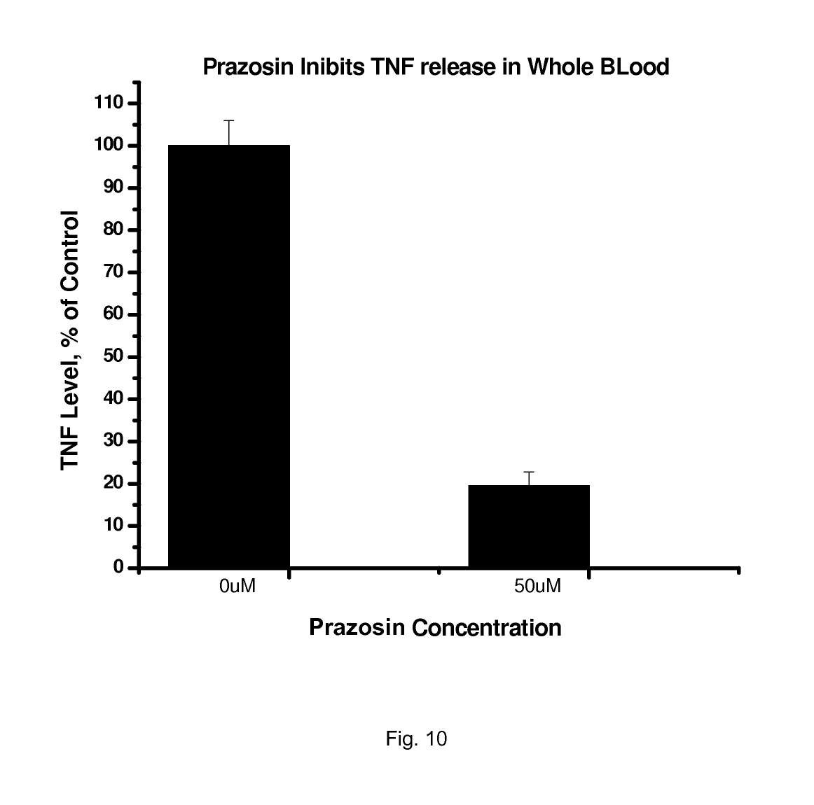

Using on our optimized whole blood assay, we tested if prazosin effect was donor specific, or gender specific. Blood was drawn from thirteen independent donors under an IRB approved protocol. Diluted blood mixed with Prazosin was incubated at 37 degrees for 2 hours. The blood samples were spun and plasma evaluated for TNF production using TNF ELISA from BD Biosciences. As shown, Prazosin reduces TNF levels in whole blood. Using prazosin treated samples, we observe nearly 100% inhibition at the upper range of the dose curve. These data suggest this compound can be used alone for the treatment of inflammation. It could also provide a possible treatment for rheumatoid arthritis. As shown in FIG. 10, Prazosin inhibits TNF production in thirteen human volunteers irrespective of the age and gender.

### Example 10

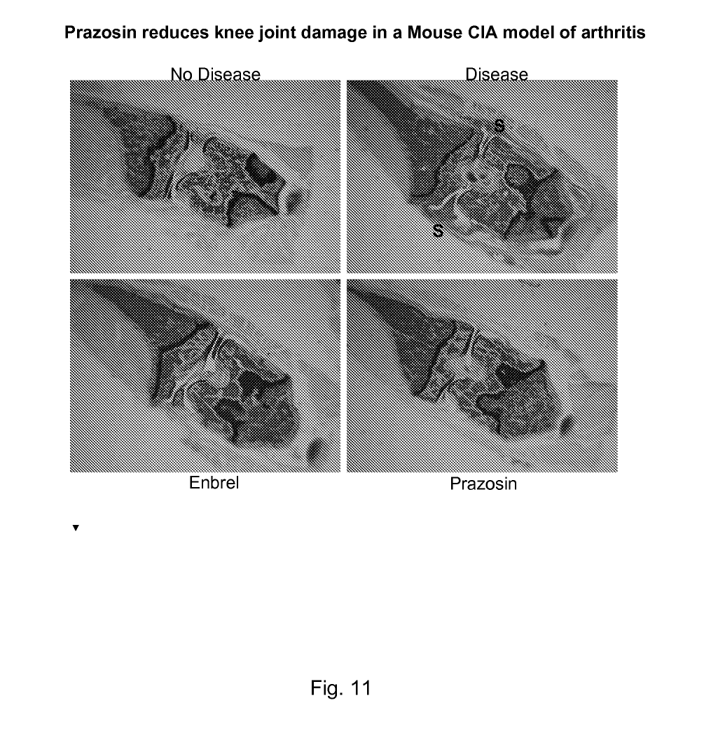

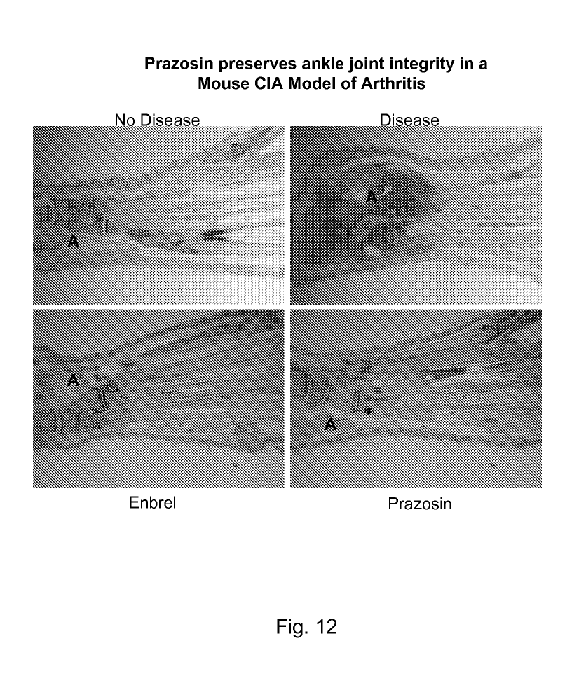

Following the CIA, limbs were stored in 10% phosphate buffered saline and subjected to histological evaluation. FIG. 11 shows the histology on the knee joints stained with Toluidine blue. FIG. 12 shows histological staining of the ankle joint. As shown, Prazosin prevents inflammation, bone and cartilage destruction, pannus formation and bone erosion.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of treating inflammation in a mammal comprising administering to the mammal a therapeutically effective amount of an alpha IA adrenergic receptor antagonist and a beta 2 adrenergic receptor antagonist effective to suppress inflammation in the mammal, the alpha IA adrenergic receptor antagonist not being an alpha adrenergic receptor 2 antagonist and being administered to the mammal without administering a beta adrenergic agonist.

2. The method of claim 1, wherein the inflammation is local or systemic.

3. The method of claim 1, the alpha IA adrenergic receptor antagonist not lowering the blood pressure of the mammal.

4. The method of claim 1, the alpha IA adrenergic receptor antagonist preventing detectable TNF production and or release.

5. The method of claim 1, the alpha IA adrenergic receptor antagonist inhibiting IL-I production and or release.

6. The method of claim 1, the alpha IA adrenergic receptor antagonist preventing joint inflammation and damage.

7. The method of claim 1, the dose of the alpha IA adrenergic receptor antagonist being about 0.001 to 100.0 mg per/Kg/day mammal body weight.

8. The method of claim 1, the inflammation being caused by an inflammatory disease selected from the group consisting of rheumatoid arthritis, rheumatoid spondylitis, osteoarthritis, other arthritic conditions, sepsis, septic-shock endotoxic shock, adult respiratory distress syndrome, chronic pulmonary inflammatory disease, silicosis, asbestosis, pulmonary sarcoidosis, bone resorption diseases, graft vs. host reactions, allograft rejections, immune deficiency syndrome (AIDS), keloid formation, scar tissue formation, Crohn's disease, fibromyalgia, ulcerative colitis, or pyresis, Multiple Sclerosis, autoimmune diabetes, systemic lupus erythematosus, asthma, xeno transplantation, chronic bronchitis, atopic dermatitis, urticaria, allergic rhinitis, allergic conjunctivitis, eosiniophilic granuloma, reperfusion injury of the myocardium and brain, chronic glomerulonephritis, Alzheimer's disease, pulmonary fibrosis, lung sarcoidosis, hepatic apoptosis, obesity, pre-eclampsia, dermal burns, cardiac arrest, congestive heart failure, myocardial infarction, acute allogenic bone transplants, and HIV viral infections.

9-23. (canceled)

24. A method of treating inflammation by inhibiting TNF and IL-I in a mammal comprising: administering to the mammal a therapeutically effective amount of an alpha IA adrenergic receptor antagonist, a beta adrenergic receptor antagonist, and a beta adrenergic receptor agonist effective to suppress inflammation in the mammal.

25. The method of claim 24, wherein the inflammation is local or systemic.

26. The method of claim 24, the alpha IA adrenergic receptor antagonist not lowering the blood pressure of the mammal.

27. The method of claim 24 the alpha IA adrenergic receptor antagonist preventing joint inflammation and damage.

28. The method of claim 24, the dose of the alpha IA adrenergic receptor antagonist being about 0.001 to 100.0 mg per/Kg/day mammal body weight.

29. The method of claim 24, the dose of the beta adrenergic receptor antagonist being about 0.001 to 100.0 mg per/Kg/day mammal body weight.

30. The method of claim 24, the dose of the beta adrenergic receptor agonist being about 0.001 to 100.0 mg per/Kg/day mammal body weight.

31. The method of claim 24, the inflammation being caused by an inflammatory disease selected from the group consisting of rheumatoid arthritis, rheumatoid spondylitis, osteoarthritis, other arthritic conditions, sepsis, septic-shock endotoxic shock, adult respiratory distress syndrome, chronic pulmonary inflammatory disease, silicosis, asbestosis, pulmonary sarcoidosis, bone resorption diseases, graft vs. host reactions, allograft rejections, immune deficiency syndrome (AIDS), keloid formation, scar tissue formation, Crohn's disease, fibromyalgia, ulcerative colitis, or pyresis, Multiple Sclerosis, autoimmune diabetes, systemic lupus erythematosus, asthma, xeno transplantation, chronic bronchitis, atopic dermatitis, urticaria, allergic rhinitis, allergic conjunctivitis, eosiniophilic granuloma, reperfusion injury of the myocardium and brain, chronic glomerulonephritis, Alzheimer's disease, pulmonary fibrosis, lung sarcoidosis, hepatic apoptosis, obesity, pre-eclampsia, dermal burns, cardiac arrest, congestive heart failure, myocardial infarction, acute allogenic bone transplants, and HIV viral infections.
